(19) United States
(12) Patent Application Publication
(10) Pub. No.: US 2008/0255091 A1
(43) Pub. Date: Oct. 16, 2008

(54) COMPOUNDS TO TREAT AMYLOIDOSIS AND PREVENT DEATH OF BETA-CELLS IN TYPE 2 DIABETES MELLITUS

(75) Inventors: Alexander B. Zolotoy, Richmond (CA); Eric S. Hayes, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: ASTRUM THERAPEUTICS PTY LTD., Melbourne (AU)

(21) Appl. No.: 12/143,691

(22) Filed: Jun. 20, 2008

Related U.S. Application Data
(63) Continuation of application No. 11/400,772, filed on Apr. 7, 2006.
(60) Provisional application No. 60/669,411, filed on Apr. 7, 2005.

(51) Int. Cl. A61K 31/55; A61K 31/165; A61K 31/421; A61K 31/18; A61K 31/4164

(52) U.S. Cl. 514/213.01; 514/616; 514/374; 514/601; 514/365

(57) ABSTRACT

The invention discloses aromatic amides and sulfonates to treat or prevent type 2 diabetes mellitus (T2DM), the pathological consequences of T2DM, to inhibit amyloidosis or to prevent death of β-cells of the pancreas.

### CROSS-REFERENCE TO RELATED APPLICATION

This application is a continuation of co-pending U.S. Utility application Ser. No. 11/400,772, filed Apr. 7, 2006, which claims the benefit under 35 U.S.C. § 119(e) of U.S. Provisional Patent Application No. 60/669,411 filed Apr. 7, 2005, which applications are incorporated herein by reference in their entireties.

### BACKGROUND OF THE INVENTION

1. Field of the Invention

The present invention discloses compounds and methods to treat patients with type 2 diabetes mellitus (T2DM). The administration of these compounds results in inhibition of amyloidosis and prevention of death of pancreatic β-cells.

2. Description of the Related Art

Islet cell amyloidosis (IA) is a basic characteristic of the pathology T2DM that is associated with the death of pancreatic β-cells (Kahn et al. Diabetes 1999, 48:241-53; Hopener et al. Mol. Cell Endocrinol. 2002, 197:205-212; O'Brien, Mol Cell Endocrinol. 2002, 197:213-219). As a consequence, β-cell mediated insulin secretion is reduced, aggravating the hyperglycemic diabetic state (Hopener et al. 2002, supra). Drugs currently available on the market do not prevent IA. A recent study in the UK analyzed the effects (11 year follow-up) of oral glycemic control agents on β-cell function and concluded that IA deposition is not diminished, and may possibly even be aggravated, and that patient β-cell function deteriorates irrespective of treatment (Turner, Diabetes Care 1998, 21:C35-C38). Thus there is a clear need for new anti-IA therapy.

The deposition of islet amyloid IA within β-cells of the pancreas is one of the main characteristics of T2DM pathology with an incidence of up to 96% (Westermark, Int J Exp Clin Invest. 1994, 1:47-60). IA has been described in humans, non-human primates and cats but is not found in rat, mouse, rabbit, hamster, hare or dog (Hopener et al. 2002, supra). In 1987 the structure of the main component of IA was determined independently by Westermark and Cooper and designated Islet Amyloid Polypeptide (IAPP) or Amylin (Cooper et al. Proc Natl Acad Sci. 1987, 84:8628-8632; Westermark et al. Proc Natl Acad Sci. 1987, 84:3881-3885). It is believed that IAPP, along with insulin and glucagon, is an active islet hormone involved in the metabolic control of glucose metabolism. IAPP is co-secreted with insulin from β-cells of the pancreas. Transformation of IAPP monomers from the alpha-helix (α-helix) to beta-sheet (β-sheet) conformation results in the formation of toxic IAPP fibrils, death of β-cells and subsequent accumulation of IA (Hopener et al. 2002, supra). Thus the transformation of IAPP impairs insulin release and aggravates the pathology of diabetes.

Reported results of studies support the relationship between IAPP, IA and β-cell death. First, in separate studies 13 of 15 (87%), 20 of 26 (77%) and 22 of 24 (92%) T2DM patients exhibited pancreatic IA compared to 0 of 10 (0%) and 1 of 14 (7%) in controls (Gebre-Medhin et al. Diabetologia 2000, 43:687-695; Clark et al. Lancet 1987, 2:231-234; Clark et al. Diabetologia 1990, 33:285-289). In human diabetics with cystic fibrosis (CF), IA incidence was reported to be 69%, compared to 17% and 0% in borderline T2DM patients and non-diabetic controls, respectively (Iannucci et al. Hum Pathol. 1984, 15:278-284). IA occupies up to 80% of islets in T2DM patients (Clark et al. Diabetes Res Clin Pract. 1995, 28:S39-S47). The density of pancreatic β-cells is decreased by 24% (P<0.05) while α-cell density increased by 58% (P<0.001) in T2DM subjects compared to controls (Gebre-Medhin et al. 2000, supra). Modest differences have also been reported in the incidence of IA in T2DM (100%) compared to control (60%) subjects (Westermark et al. Diabetologia 1978, 15:417-421). However, the volume of islets completely free from amyloid in diabetic subjects was 0.41±0.03 cm³ compared to 1.58±0.16 cm³ in non-diabetics subjects (P<0.05).

Second, in humans the presence of IAPP-induced IA is associated with the loss of 24% to 50% of pancreatic β-cells (Clark et al. Diabetes Res. 1988, 9:151-159; Couce et al. J Clin Endocrinol Metab. 1996, 81:1267-1272). This conclusion was confirmed with respect to differentiation between obese and lean human subjects (Butler et al. Diabetes 2003, 52:2304-2314). Obese subjects with T2DM exhibited a 63% deficit in relative β-cell volume compared to non-diabetic obese subjects (P<0.01), whereas lean subjects with T2DM exhibited a 41% decrease in relative β-cell volume compared to non-diabetic lean controls (P<0.05). The observed decreased β-cell volume in patients with T2DM was due to a specific decrease in the number of β-cells rather than a generalized decrease of total cell volume. The frequency of apoptotic (cell-death) events (frequency of β-cell apoptosis/relative volume of β-cells) was 3 times higher in obese subjects with T2DM compared to obese controls (P<0.05) and 10 times higher in lean T2DM subjects compared to lean controls (P<0.05). IA has been observed in 81% of obese T2DM cases compared to 10% in obese controls (P<0.01) and in 88% of lean T2DM cases compared to 13% of lean controls (P<0.01). The frequency and extent of Congo red birefringence of islets (visual measure of IAPP fibril formation and IA deposition) was also significantly higher in obese and lean T2DM patients compared to appropriate controls.

Finally, similar links between IAPP, IA and β-cell death have been demonstrated in various animal models of T2DM. In cats the presence of IA is associated with the loss of up to 50% of β-cells (O'Brien et al. J Comp Pathol. 1986, 96:357-359). Obese non-transgenic mice do not develop diabetes. They adapt to insulin resistance through a 9-fold increase (P<0.001) in β-cell mass that results from a 1.7-fold increase in islet neogenesis (P<0.05) and a 5-fold increase in β-cell replication per islet (P<0.001) compared to non-obese controls. Obese transgenic mice expressing the human IAPP (hIAPP) gene develop midlife diabetes with islet amyloid and an 80% (P<0.001) decrease in β-cell mass that is not compensated for. The mechanism subserving the failed expansion was a 10-fold increase in β-cell apoptosis compared to controls (P<0.001). The frequency of β-cell apoptosis correlates with the rate of increase of IAPP fibril formation and IA, but not to the extent of islet amyloid or the blood glucose concentration (Butler et al. 2003, supra). Additional studies with hIAPP transgenic mice have demonstrated that amyloid severity (amyloid area/islet area) inversely correlates with viable β-cell densities (r=−0.59, P<0.0001) (Wang et al. Diabetes 2001, 50:2514-2520).

The development of IA correlates with the development of T2DM. Studies with Macaca nigra, a species of old world monkey that develops spontaneous IA and T2DM, have shown that initially IA reduces insulin secretion associated with mild impairments of glucose tolerance without changes in fasting glucose concentrations. Long term studies in the same species indicated continued IA associated with a further reduced insulin secretion profile and deterioration of glucose tolerance. The development of fasting hyperglycemia was a late occurring phenomenon and appeared in animals with substantial IA (Howard C F, Jr. Diabetologia 1986, 29:301-306). Macaca mulatta were followed during an entire life span and post-mortem pancreatic tissue from 26 monkeys were examined (de Koning et al. Diabetologia 1993, 36:378-384). Four groups of animals were studied: group I, young (<10 years), lean and normoglycemic; group II, older (>10 years), lean or obese, normoglycemic; group III, normoglycemic and hyperinsulinemic; and group IV, diabetic. Islet sizes were larger in animals from groups III (P<0.01) and IV (P<0.0001) compared to groups I and II. Amyloid was absent in group I (0%), but small deposits were present in 3 of 9 group II animals (33%) and in 4 of 6 group III animals (75%) and occupied between 0.03% and 45% of the islet area. Amyloid was present in 8 of 8 group IV animals (100%) and occupied between 37% and 81% of islet area. Every islet was affected in 7 of 8 diabetic monkeys (88%). It was concluded that islet amyloid appears to precede the development of overt diabetes in Macaca mulatta and is likely to be a factor in the destruction of islet cells and onset of hyperglycemia. IA has also been demonstrated in 79% of diabetic cats, 44% of cats with impaired glucose tolerance and 25% of normal cats (Johnson K H et al. Am. J. Pathol. 1989, 135:245-250). IAPP immunoreactivity was very low in 8 of 8 diabetic cats, was increased in 6 of 6 cats with impaired glucose tolerance and was highest in normal cats. The investigators concluded that the presence of IA and the disappearance of IAPP from β-cell loss predicted impaired glucose tolerance with a probability of 88%.

Transformation of IAPP from the α-helix conformation into the β-sheet conformation results in the formation of toxic IAPP fibrils, IA and the death of pancreatic β-cells. At a concentration of 5 μM and higher, IAPP is stabilized in the fibril form and induces beta-cell death; whereas at 1 μM and lower, IAPP is not in a fibrillogenic form and does not induce beta-cell death. Researchers noted the following manifestations of cytotoxicity: plasma membrane blebbing, inappropriate chromatin condensation and DNA fragmentation (Lorenzo et al. Nature. 1994, 368: 756-760). Recent studies that employed better protein production and stabilization procedures have found the EC₅₀ for IAPP-mediated β-cell cytotoxicity to be approximately 100 nM and confirmed that the cytotoxic form was the fibrillar form of the peptide (Krampert et al. Chem Biol. 2000, 7:855-71). Application of fibrillogenic human IAPP to pure planar lipid bilayer membranes dramatically increases membrane conductance, whereas the application of not-fibrillogenic rat IAPP has no effect on conductance (Mirzabekov et al. J Biol Chem. 1996, 271:1988-1992). Increases in membrane conductance (e.g., influx of Ca⁺² and Na⁺ and efflux of K⁺) inevitably leads to cytotoxicity. Independent studies have demonstrated that human IAPP induces apoptosis in rat RINm5F cells (Zhang et al. FEBS Lett. 1999, 455:315-320; Saafi et al. Cell Biol Int. 2001, 25:339-350). Membrane blebbing and microvilli loss were the earliest detectable apoptosis-related phenomena, evident as early as 1 hour after hIAPP exposure. Following 6 to 12 hours of human IAPP-treatment, chromatin margination became evident, consistent with detection of DNA laddering at the same time. Nuclear shrinkage, nuclear membrane convolution and prominent cytoplasmic vacuolization were clearly recognized at 22 hours post-treatment.

Thus the development of anti-amyloidosis agents that are capable of preventing death of pancreatic β-cells remains an unmet medical need. Anti-amyloidosis agents for type 2 diabetes mellitus have been reported. α-Amino-γ-sulfonate and α-amino-δ-sulfonate derivatives are disclosed in U.S. Pat. No. 6,562,836, while alky sulfate and sulfonate derivatives are disclosed in U.S. Pat. Nos. 5,972,328 and 5,728,375. Bis- and tris-dihydroxyaryl compounds and their methylenedioxy analogs are disclosed in PCT Patent Application WO 03/101927 A1. Glucose pentasulfate is disclosed in U.S. Pat. No. 6,037,327. Derivatives of 1,2,3,4-tetrahydroisochinoline are disclosed in PCT Patent Application WO 00/71101 A2. It was shown that anti-amyloidosis agents such as Congo Red (Lorenzo et al. Proc Natl Acad Sci. 1994, 91:12243-12247) and the peptide SNNFGA (Scrocchi et al. J Mol Biol. 2002, 318:697-706) completely or partially prevented β-cell death induced by a fibrillogenic form of IAPP. The main disadvantages with respect to the disclosed compounds are: a) potency was not reported (U.S. Pat. No. 6,562,836, PCT Patent Application No. WO 00/71101 A2) or was low, between 1 and 20 mM (U.S. Pat. Nos. 5,972,328, 6,037,327); b) no report of the effect of compounds on β-cells; and c) the effectiveness of compounds in vivo has not been described for any of the previously disclosed compounds.

Accordingly, there is a need for compounds and compositions to treat or prevent T2DM.

### BRIEF SUMMARY OF THE INVENTION

As disclosed in certain embodiments of the present invention, compounds of formulas I-XXIII, including compounds I, III, and XXIII, inhibit amyloidosis, prevent death of pancreatic β-cells and thus may be useful for treating or preventing T2DM. In certain embodiments, pharmaceutical compositions for use in the treatment or prevention of type 2 diabetes mellitus (T2DM), pathological consequences of T2DM, or inhibition of IAPP-induced amyloidosis, or in the prevention of death of pancreatic β-cells comprise a pharmaceutical carrier, diluent or excipient and a compound of any of formulas I-XXIII. Compounds of formulas I-XXIII include the following:

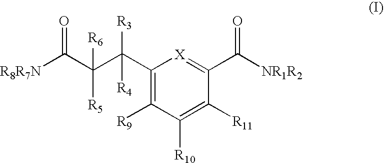

wherein X is C—H fragment or nitrogen;

R₁, R₂, R₇, R₈ are independently selected from hydrogen and C₁-C₃ alkyl;

R₃, R₄, R₅, R₆ are independently selected from hydrogen, methyl, ethyl and propyl;

R₉, R₁₀, R₁₁ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

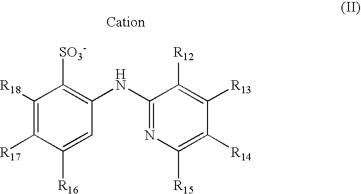

wherein R₁₂, R₁₃, R₁₄, R₁₅, R₁₆, R₁₇, R₁₈ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

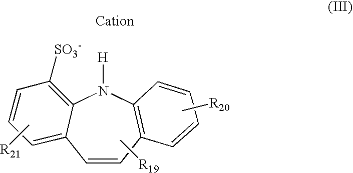

wherein R₁₉, R₂₀, R₂₁ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

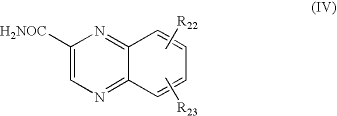

wherein R₂₂ and R₂₃ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

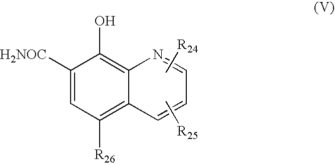

wherein R₂₄, R₂₅, R₂₆ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

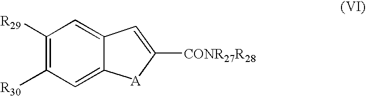

wherein A is selected from oxygen, sulfur and NR₄₀ wherein R₄₀ is selected from hydrogen and C₁-C₆ alkyl;

R₂₇ and R₂₈ are independently selected from hydrogen and C₁-C₆ alkyl;

R₂₉ and R₃₀ are independently selected from hydrogen, methyl, chlorine, bromine and fluorine;

with the proviso that, where R₄₀ is C₁-C₆ alkyl, then either R₂₇ or R₂₈ is hydrogen;

with the further proviso that aromatic carbon atoms may be optionally replaced by aromatic nitrogen atoms.

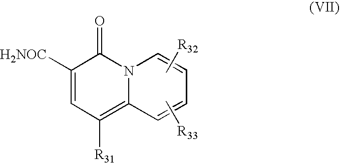

wherein R₃₁, R₃₂ and R₃₃ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

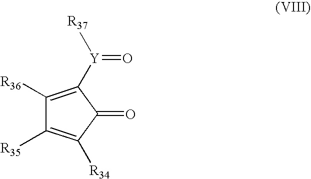

wherein R₃₄, R₃₅, R₃₆ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl;

with the proviso that R₃₅ and R₃₆ may be optionally connected to form a bicyclic system wherein R₃₅ and R₃₆ together are represented by —CH═CR₄₀—CH═CH—, —CH═CH—CR₄₀═CH—, —N═CR₄₀—CH═CH—, —N═CH—CR₄₀═CH—, —CH═N—CR₄₀═CH—, —CH═CR₄₀—N═CH—, —CH═CR₄₀—CH═N—, —CH═CH—CR₄₀═N—, —X₁—CR₄₀═CH—X₂—, —X₁—CH═CR₄₀—X₂—, —X₁—CH═CR₄₀—, —CR₄₀═CH—X₁—, —CH₂—CH₂—CH₂—CH₂—, —CH₂—CH₂—X₁—CH₂—, —CH₂—X₁—CH₂—CH₂—, —X₁—CH₂—CH₂—X₂—, —CH₂—CH₂—CH₂—, —X₁—CH₂—CH₂—, —CH₂—X₁—CH₂—, or —CH₂—CH₂—X₁—;

wherein X₁ and X₂ are independently selected from oxygen, sulfur and NR₃₈;

R₄₀ is selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl;

Y is selected from carbon and S═O;

R₃₇ is selected from C₁-C₆ alkyl, NH(C₁-C₆ alkyl) and phenyl wherein phenyl may be optionally substituted by bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) or N(R₃₈R₃₉) wherein R₃₈ and R₃₉ are independently selected from hydrogen, acetyl, methanesulfonyl and C₁-C₆-alkyl;

with the proviso that endocyclic carbon atoms may be optionally replaced by nitrogen atoms; certain embodiments of such compounds are represented by formulas IX-XIII;

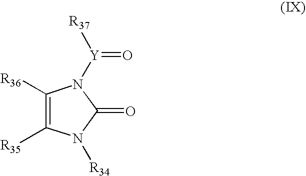

wherein R₃₄, R₃₅, R₃₆, R₃₇ and Y have the same assignations as for the formula VIII;

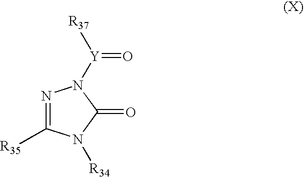

wherein R₃₄, R₃₅, R₃₇ and Y have the same assignations as for formula VIII;

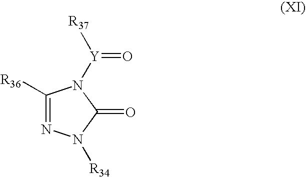

wherein R₃₄, R₃₆, R₃₇ and Y have the same assignations as for formula VIII;

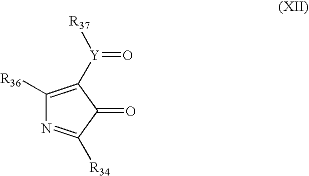

wherein R₃₄, R₃₆, R₃₇ and Y have the same assignations as for formula VIII;

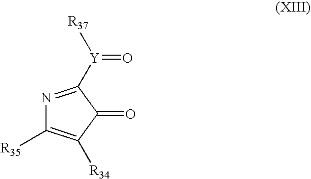

wherein R₃₄, R₃₅, R₃₇ and Y have the same assignations as for formula VIII;

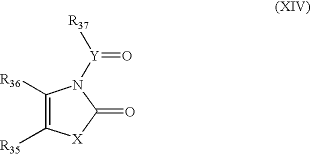

wherein X is selected from oxygen and sulfur;

R₃₅, R₃₆, R₃₇ and Y have the same assignations as for formula VIII;

with the proviso that endocyclic carbon atoms may be optionally replaced by nitrogen atoms; certain embodiments of such compounds are represented by formulas XV and XVI;

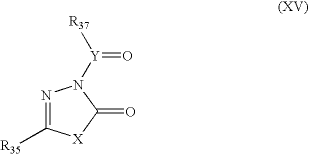

wherein X is selected from oxygen and sulfur;

R₃₅, R₃₇ and Y have the same assignations as for formula VIII;

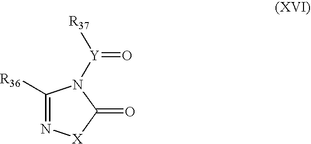

wherein X is selected from oxygen and sulfur;

R₃₆, R₃₇ and Y have the same assignations as for formula VIII;

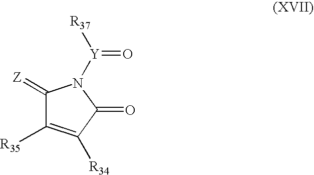

wherein Z is selected from oxygen, sulfur or CR₄₁R₄₂, wherein R₄₁ and R₄₂ are independently selected from hydrogen, methyl or phenyl, wherein phenyl may be optionally substituted by bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) or N(R₃₈R₃₉) wherein R₃₈ and R₃₉ are independently selected from hydrogen, acetyl, methanesulfonyl and C₁-C₆-alkyl;

R₃₄, R₃₅, R₃₇ and Y have the same assignations as for formula VIII;

with the proviso that endocyclic carbon atoms may be replaced by nitrogen; certain embodiments of such compounds are represented by the formulas XVIII and XIX;

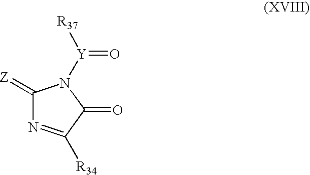

wherein R₃₄, R₃₇, Z and Y are assigned as for formula XVII;

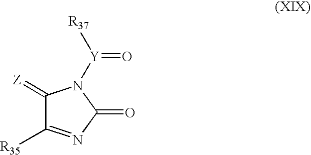

wherein R₃₅, R₃₇, Z and Y are assigned as for formula XVII;

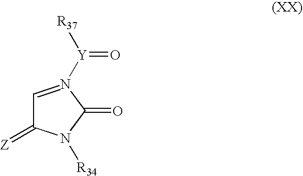

wherein Z, R₃₄, R₃₆, R₃₇, and Y have the same assignations as in formula XVII;

with the proviso that endocyclic carbon atoms may be replaced by the nitrogen; an embodiment of such compounds is represented by the formula XXI;

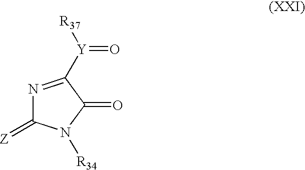

wherein Z, R₃₄, R₃₇ and Y have the same assignations as in formula XVII;

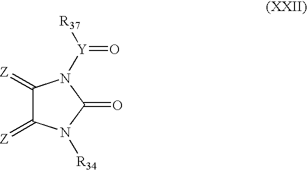

wherein Z, R₃₄, R₃₇ and Y have the same assignations as in formula XVII.

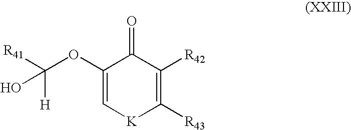

wherein R₄₁ is selected from CF₃, C₂F₅, and C₃F₇;

R₄₂ and R₄₃ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl;

K is selected from oxygen, sulfur, NR₄₄ and C═CR₄₆R₄₇ wherein R₄₄ is selected from hydrogen, acetyl, methanesulfonyl and C₁-C₆-alkyl; R₄₆ and R₄₇ are independently selected from hydrogen, methyl and phenyl, wherein phenyl may be substituted by bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) or N(R₃₈R₃₉) wherein R₃₈ and R₃₉ are independently selected from hydrogen, acetyl, methanesulfonyl and C₁-C₆-alkyl;

with the proviso that R₄₃ and R₄₄ may be optionally connected to form a bicyclic system wherein R₄₃ and R₄₄ together are represented by —CH═CR₄₅—CH═CH—, —CH═CH—CR₄₅═CH—, —N═CR₄₅—CH═CH—, —N═CH—CR₄₅═CH—, —CH═N—CR₄₅═CH—, —CH═CR₄₅—N═CH—, —CH═CR₄₅—CH═N—, —CH═CH—CR₄₅═N—, —X₁—CR₄₅═CH—X₂—, —X₁—CH═CR₄₅—X₂—, —X₁—CH═CR₄₅—, —CR₄₅═CH—X₁—, —CH₂—CH₂—CH₂—CH₂—, —CH₂—CH₂—X₁—CH₂—, —CH₂—X₁—CH₂—CH₂—, —X₁—CH₂—CH₂—X₂—, —CH₂—CH₂—CH₂—, —X₁—CH₂—CH₂—, —CH₂—X₁—CH₂—, or —CH₂—CH₂—X₂—;

wherein X₁ and X₂ are independently selected from oxygen, sulfur and NR₃₈;

R₄₀ is selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl;

In certain embodiments, pharmaceutical compositions for use in the treatment or prevention of type 2 diabetes mellitus (T2DM), pathological consequences of T2DM, or inhibition of IAPP-induced amyloidosis, or in the prevention of death of pancreatic β-cells comprise a pharmaceutical carrier, diluent or excipient and a compound of any of formulas Ia-VIIa, Ib, IIb, IXa, IXb, XIVa, XIVb, and XXIIIa:

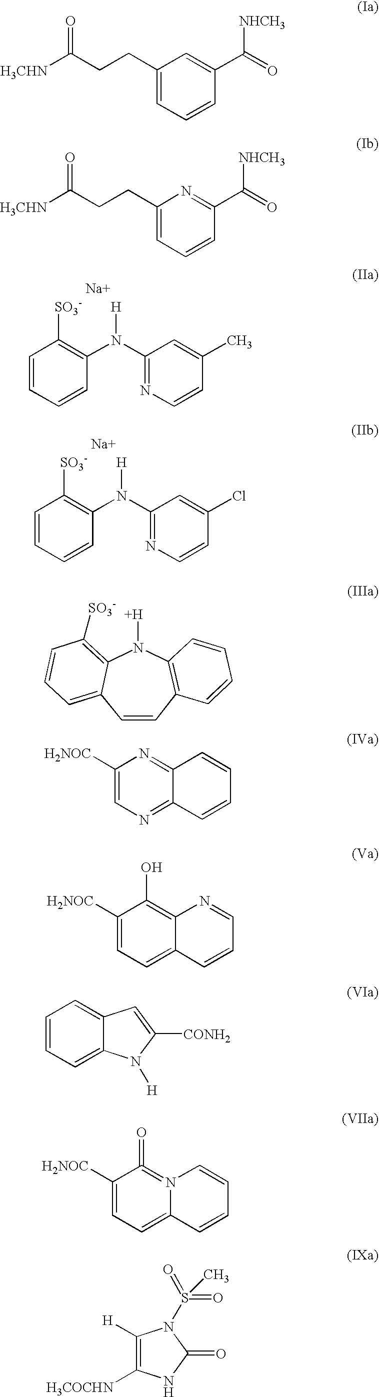

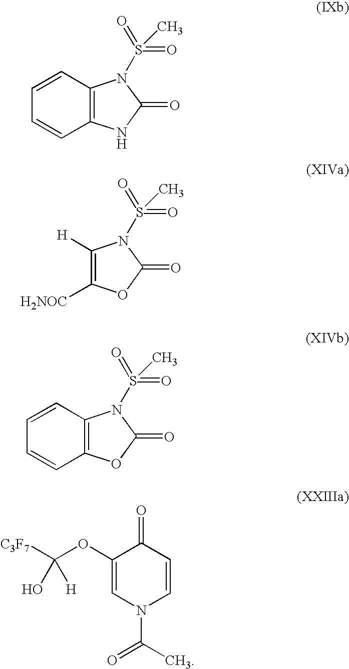

In certain embodiments, the present invention includes compounds I, III, and XXIII:

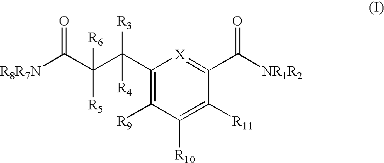

wherein X is C—H fragment or nitrogen;

R₁, R₂, R₇, R₈ are independently selected from hydrogen and C₁-C₃ alkyl;

R₃, R₄, R₅, R₆ are independently selected from hydrogen, methyl, ethyl and propyl;

R₉, R₁₀, R₁₁ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl; and

with the proviso that aromatic carbon atoms may be optionally replaced by aromatic nitrogen atoms;

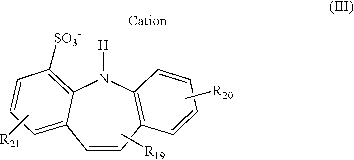

wherein R₁₉, R₂₀, R₂₁ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl; and

with the proviso that aromatic carbon atoms may be optionally replaced by aromatic nitrogen atoms;

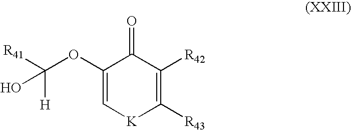

wherein R₄₁ is selected from CF₃, C₂F₅ and C₃F₇;

R₄₂ and R₄₃ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl;

K is selected from oxygen, sulfur, NR₄₄ and C═CR₄₆R₄₇ wherein R₄₄ is hydrogen, acetyl, methanesulfonyl and C₁-C₆-alkyl; R₄₆ and R₄₇ are independently selected from hydrogen, methyl and phenyl, wherein phenyl may be substituted by bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) or N(R₃₈R₃₉) wherein R₃₈ and R₃₉ are independently selected from hydrogen, acetyl, methanesulfonyl and C₁-C₆-alkyl;

with the proviso that R₄₃ and R₄₄ may be optionally connected to form a bicyclic system wherein R₄₃ and R₄₄ together are represented by —CH═CR₄₅—CH═CH—, —CH═CH—CR₄₅═CH—, —N═CR₄₅—CH═CH—, —N═CH—CR₄₅═CH—, —CH═N—CR₄₅═CH—, —CH═CR₄₅—N═CH—, —CH═CR₄₅—CH═N—, —CH═CH—CR₄₅═N—, —X₁—CR₄₅═CH—X₂—, —X₁—CH═CR₄₅—X₂—, —X₁—CH═CR₄₅—, —CR₄₅═CH—X₁—, —CH₂—CH₂—CH₂—CH₂—, —CH₂—CH₂—X₁—CH₂—, —CH₂—X₁—CH₂—CH₂—, —X₁—CH₂—CH₂—X₂—, —CH₂—CH₂—CH₂—, —X₁—CH₂—CH₂—, —CH₂—X₁—CH₂—, or —CH₂—CH₂—X₂—; and

wherein X₁ and X₂ are independently oxygen, sulfur and NR₃₈; R₄₀ is selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl.

In certain embodiments, the present invention provides compounds I-XXIII, Ia-VIIa, Ib, IIb, IXa, IXb, XIVa, XIVb, and XXIIIa, presented above, or compositions comprising compounds I-XXIII, Ia-VIIa, Ib, IIb, IXa, IXb, XIVa, XIVb, or XXIIIa for use in a method for the treatment or prevention of T2DM, pathological consequences of T2DM, or IAPP-induced amyloidosis, or prevention of death of pancreatic β-cells.

In certain other embodiments, the present invention provides compounds I-XXIII, Ia-VIIa, Ib, IIb, IXa, IXb, XIVa, XIVb, and XXIIIa, or compositions comprising compounds I-XXIII, Ia-VIIa, Ib, IIb, IXa, IXb, XIVa, XIVb, or XXIIIa for use in the preparation of a medicament for the treatment or prevention of T2DM, pathological consequences of T2DM, or IAPP-induced amyloidosis, or prevention of death of pancreatic β-cells. In certain embodiments, the compound or composition may further include a pharmaceutical carrier, diluent or excipient.

In yet other embodiments, the present invention provides methods for the treatment or prevention of T2DM, pathological consequences of T2DM, or IAPP-induced amyloidosis, or prevention of death of pancreatic β-cells by administering to a warm-blooded animal, including humans, in need thereof a therapeutically effective amount of a compound selected from compounds I-XXIII, Ia-VIIa, Ib, IIb, IXa, IXb, XIVa, XIVb, and XXIIIa, or a pharmaceutical composition thereof.

Compounds of formulas I-XXIII, Ia-VIIa, Ib, IIb, IXa, IXb, XIVa, XIVb, and XXIIIa may be used in free or solvated form or as a pharmaceutically acceptable salt thereof and include isolated enantiomeric, diastereomeric and geometric isomers thereof, metabolites, metabolic precursors or prodrugs in crystalline, or amorphous, or liquid or gel forms including all polymorphic modifications thereof.

These and other aspects of the present invention will become apparent upon reference to the following detailed description. All references disclosed here are hereby incorporated by reference in their entirety as if each were incorporated individually.

### DETAILED DESCRIPTION OF INVENTION

As used herein, the following terms are defined as follows:

“Alkyl” refers to a branched or unbranched hydrocarbon fragment containing the specified number of carbon atoms and having one point of attachment. Examples include n-propyl (a C₃ alkyl), isopropyl (also a C₃ alkyl) and t-butyl (a C₄ alkyl).

“Alkoxyalkyl” refers to an alkylene group substituted with an alkoxy group. For example methyloxyethyl (CH₃OCH₂CH₃—) and ethoxymethyl (CH₃CH₂OCH₂—) are both C₃ alkoxyalkyl groups.

“Alkanoyloxy” refers to an ester substituent wherein the ether oxygen is the point of attachment to the molecule. Examples include propanoyloxy (CH₃CH₂C(O)—O—), a C₃ alkanoyloxy and ethanoyloxy (CH₃C(O)—O—), a C₂ alkanoyloxy.

“Alkoxy” refers to an O-atom substituted by an alkyl group, for example methoxy (—OCH₃) a C₁ alkoxy.

“Alkoxycarbonyl” refers to an ester substituent wherein the carbonyl group is the point of attachment to the molecule. Examples include ethoxycarbonyl (CH₃CH₂OC(O)—), a C₃ alkoxycarbonyl and methoxycarbonyl (CH₃OC(O)—), a C₂ alkoxycarbonyl.

“Aryl” refers to aromatic groups which have at least one ring having a conjugated pi electron system and includes carbocyclic aryl, heterocyclic aryl (also known as heteroaryl groups) and biaryl groups, all of which may be optionally substituted.

“Thioalkyl” refers to a sulfur atom substituted by an alkyl group, for example thiomethyl (CH₃S—), a C₁ thioalkyl.

Certain compounds of the present invention or for use in the pharmaceutical compositions or methods of the present invention are represented by formulas I-XXIII:

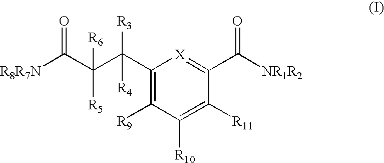

wherein X is C—H fragment or nitrogen;

R₁, R₂, R₇, R₈ are independently selected from hydrogen and C₁-C₃ alkyl;

R₃, R₄, R₅, R₆ are independently selected from hydrogen, methyl, ethyl and propyl;

R₉, R₁₀, R₁₁ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

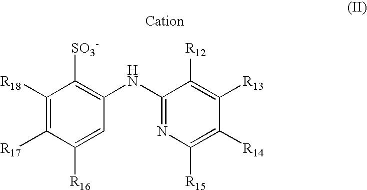

wherein R₁₂, R₁₃, R₁₄, R₁₅, R₁₆, R₁₇, R₁₈ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

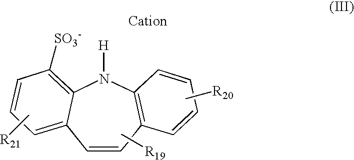

wherein R₁₉, R₂₀, R₂₁ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl aryl, CON(R₃₈R₃₉)N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

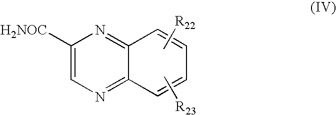

wherein R₂₂ and R₂₃ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

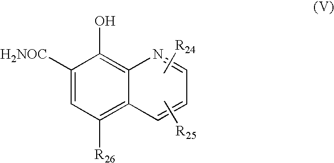

wherein R₂₄, R₂₅, R₂₆ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

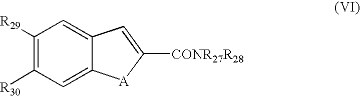

wherein A is selected from oxygen, sulfur and NR₄₀ wherein R₄₀ is selected from hydrogen or C₁-C₆ alkyl;

R₂₇ and R₂₈ are independently selected from hydrogen and C₁-C₆ alkyl;

R₂₉ and R₃₀ are independently selected from hydrogen, methyl, chlorine, bromine, and fluorine;

with the proviso that, where R₄₀ is C₁-C₆ alkyl, then either R₂₇ or R₂₈ is hydrogen;

with the further proviso that aromatic carbon atoms may be optionally replaced by aromatic nitrogen atoms.

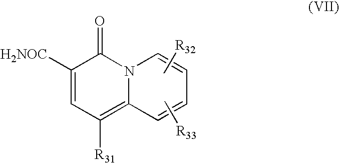

wherein R₃₁, R₃₂ and R₃₃ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl;

with the proviso that aromatic carbon atoms may be optionally replaced by aromatic nitrogen atoms.

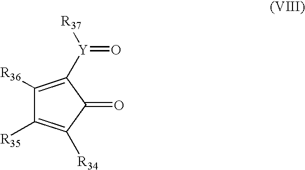

wherein R₃₄, R₃₅, R₃₆ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl;

with the proviso that R₃₅ and R₃₆ may be optionally connected to form a bicyclic system wherein R₃₅ and R₃₆ together are represented by —CH═CR₄₀—CH═CH—, —CH═CH—CR₄₀═CH—, —N═CR₄₀—CH═CH—, —N═CH—CR₄₀═CH—, —CH═N—CR₄₀═CH—, —CH═CR₄₀—N═CH—, —CH═CR₄₀—CH═N—, —CH═CH—CR₄₀═N—, —X₁—CR₄₀═CH—X₂—, —X₁—CH═CR₄₀—X₂—, —X₁—CH═CR₄₀—, —CR₄₀═CH—X₁—, —CH₂—CH₂—CH₂—CH₂—, —CH₂—CH₂—X₁—CH₂—, —CH₂—X₁—CH₂—CH₂—, —X₁—CH₂—CH₂—X₂—, —CH₂—CH₂—CH₂—, —X₁—CH₂—CH₂—, —CH₂—X₁—CH₂—, or —CH₂—CH₂—X₁—;

wherein X₁ and X₂ are independently selected from oxygen, sulfur and NR₃₈;

R₄₀ is selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl;

Y is selected from carbon and S═O;

R₃₇ is selected from C₁-C₆ alkyl, NH(C₁-C₆ alkyl) and phenyl wherein phenyl may be optionally substituted by bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) or N(R₃₈R₃₉) wherein R₃₈ and R₃₉ are independently selected from hydrogen, acetyl, methanesulfonyl and C₁-C₆-alkyl;

with the proviso that endocyclic carbon atoms may be optionally replaced by nitrogen atoms; certain embodiments of such compounds are represented by formulas IX-XIII;

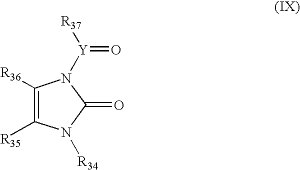

wherein R₃₄, R₃₅, R₃₆, R₃₇ and Y have the same assignations as for the formula VIII;

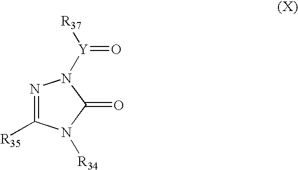

wherein R₃₄, R₃₅, R₃₇ and Y have the same assignations as for formula VIII;

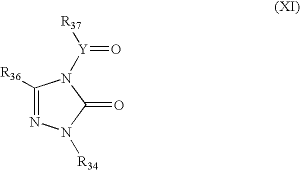

wherein R₃₄, R₃₆, R₃₇ and Y have the same assignations as for formula VIII;

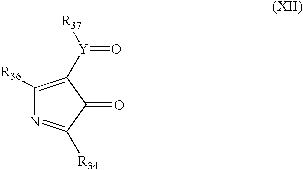

wherein R₃₄, R₃₆, R₃₇ and Y have the same assignations as for formula VIII;

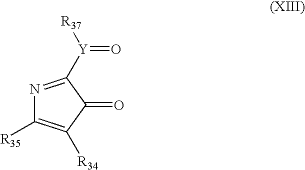

wherein R₃₄, R₃₅, R₃₇ and Y have the same assignations as for formula VIII;

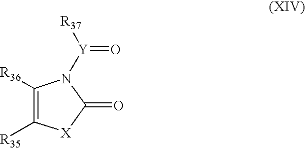

wherein X is selected from oxygen and sulfur;

R₃₅, R₃₆, R₃₇ and Y have the same assignations as for formula VIII;

with the proviso that endocyclic carbon atoms may be optionally replaced by nitrogen atoms; certain embodiments of such compounds are represented by formulas XV and XVI;

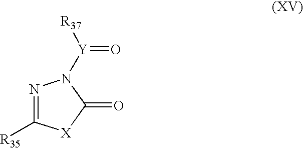

wherein X is selected from oxygen and sulfur;

R₃₅, R₃₇ and Y have the same assignations as for formula VIII;

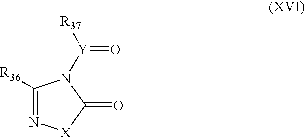

wherein X is selected from oxygen and sulfur;

R₃₆, R₃₇ and Y have the same assignations as for formula VIII;

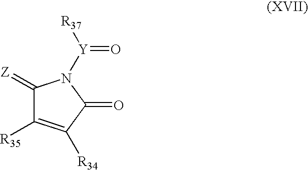

wherein Z is selected from oxygen, sulfur and CR₄₁R₄₂, wherein R₄₁ and R₄₂ are independently selected from hydrogen, methyl or phenyl, wherein phenyl may be optionally substituted by bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) or N(R₃₈R₃₉) wherein R₃₈ and R₃₉ are independently selected from hydrogen, acetyl, methanesulfonyl and C₁-C₆-alkyl;

R₃₄, R₃₅, R₃₇ and Y have the same assignations as for formula VIII;

with the proviso that endocyclic carbon atoms may be replaced by nitrogen; certain embodiments of such compounds are represented by the formulas XVIII and XIX;

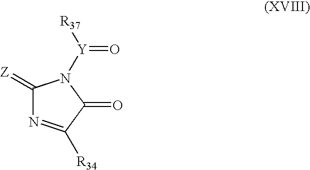

wherein R₃₄, R₃₇, Z and Y are assigned as for formula XVII;

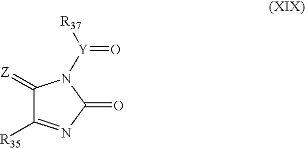

wherein R₃₅, R₃₇, Z and Y are assigned as for formula XVII; —CR₄₅═CH—X₁—, —CH₂—CH₂—CH₂—CH₂—, —CH₂—CH₂—X₁—CH₂—, —CH₂—X₁—CH₂—CH₂—, —X₁—CH₂—CH₂—X₂—, —CH₂—CH₂—CH₂—, —X₁—CH₂—CH₂—, —CH₂—X₁—CH₂—, or —CH₂—CH₂—X₂—;

wherein X₁ and X₂ are independently selected from oxygen, sulfur and NR₃₈;

R₄₀ is selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl;

Compounds of formulas I-XXIII may be used in free or solvate form or in pharmaceutically acceptable salt thereof and include isolated enantiomeric, diastereomeric and geometric isomers thereof, metabolites, metabolic precursors or prodrugs in crystalline, or amorphous, or liquid or gel forms including all polymorphic modifications thereof.

One preferred embodiment of the present invention is a compound of formula Ia with the following structure:

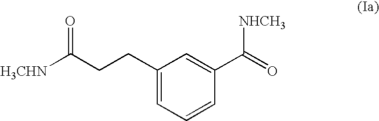

Another preferred embodiment of the present invention is a compound of formula Ib with the following structure:

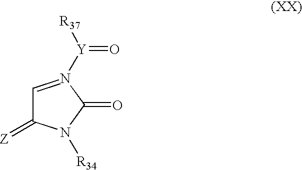

wherein Z, R₃₄, R₃₆, R₃₇, and Y have the same assignations as in formula XVII;

with the proviso that endocyclic carbon atom my be replaced by the nitrogen; an embodiment of such a compound is represented by the formula XXI;

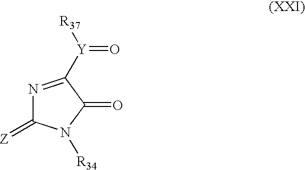

wherein Z, R₃₄, R₃₇ and Y have the same assignations as in formula XVII;

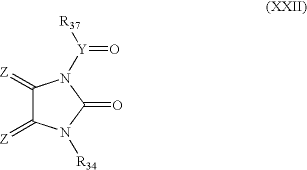

wherein Z, R₃₄, R₃₇ and Y have the same assignations as in formula XVII.

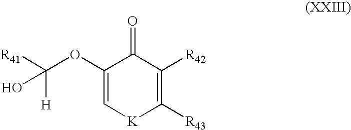

wherein R₄₁ is selected from CF₃, C₂F₅, and C₃F₇;

R₄₂ and R₄₃ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆-alkyl;

K is selected from oxygen, sulfur, NR₄₄ and C═CR₄₆R₄₇ wherein R₄₄ is selected from hydrogen, acetyl, methanesulfonyl and C₁-C₆-alkyl; R₄₆ and R₄₇ are independently selected from hydrogen, methyl or phenyl, wherein phenyl may be substituted by bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) or N(R₃₈R₃₉) wherein R₃₈ and R₃₉ are independently selected from hydrogen, acetyl, methanesulfonyl and C₁-C₆-alkyl;

with the proviso that R₄₃ and R₄₄ may be optionally connected to form a bicyclic system wherein R₄₃ and R₄₄ together are represented by —CH═CR₄₅—CH═CH—, —CH═CH—CR₄₅═CH—, —N═CR₄₅—CH═CH—, —N═CH—CR₄₅═CH—, —CH═N—CR₄₅═CH—, —CH═CR₄₅—N═CH—, —CH═CR₄₅—CH═N—, —CH═CH—CR₄₅═N—, —X₁—CR₄₅═CH—X₂—, —X₁—CH═CR₄₅—X₂—, —X₁—CH═CR₄₅—,

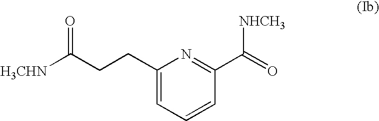

Another preferred embodiment of the present invention is a compound of formula IIa with the following structure:

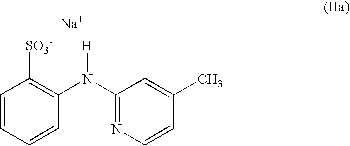

Another preferred embodiment of the present invention is a compound of formula IIb with the following structure:

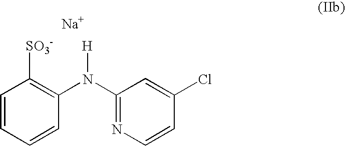

Another preferred embodiment of the present invention is a compound of formula IIIa with the following structure:

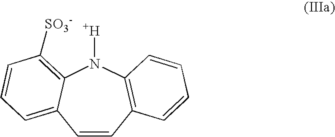

Another preferred embodiment of the present invention is a compound of formula IVa with the following structure:

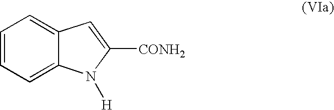

Another preferred embodiment of the present invention is a compound of formula Va with the following structure:

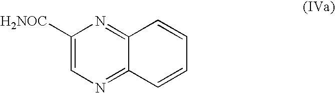

Another preferred embodiment of the present invention is a compound of formula VIa with the following structure:

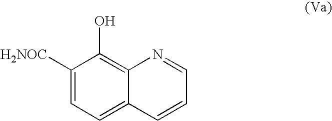

Another preferred embodiment of the present invention is a compound of formula VIIa with the following structure:

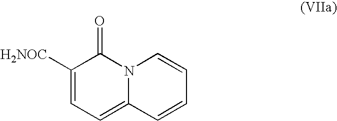

Another preferred embodiment of the present invention is a compound of formula IXa with the following structure:

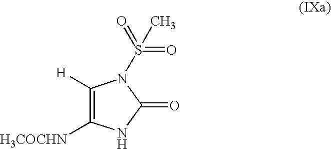

Another preferred embodiment of the present invention is a compound of formula IXb with the following structure

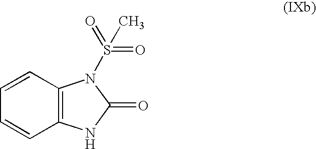

Another preferred compound of the present invention is a compound of formula XIVa with the following structure

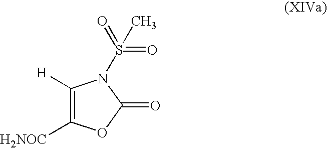

Another preferred embodiment of the present invention is a compound of formula XIVb with the following structure

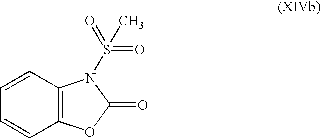

Another preferred embodiment of the present invention is a compound of formula XXIIIa with the following structure

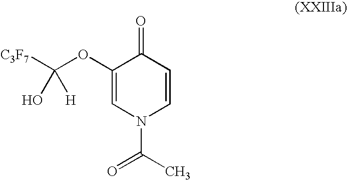

As disclosed in certain embodiments of the present invention, compounds of formulas I-XXIII, including compounds I, III, and XXIII, may be useful for treating and/or preventing T2DM and pathological consequences of T2DM in warm-blooded animals, including humans.

In certain embodiments the present invention provides compounds of formulas I-XXIII to inhibit IAPP-induced amyloidosis.

In further embodiments the present invention provides compounds of formula I-XXIII to prevent death of pancreatic β-cell.

In other embodiments the present invention provides a method for the treatment and/or prevention of T2DM and its pathological consequences, which comprises administering to a warm-blooded animal including human in need thereof a therapeutically effective amount of compounds of formulas I-XXIII.

In yet other embodiments the present invention provides a method for inhibition of amyloidosis and prevention of pancreatic β-cell death, which comprises administering to a warm-blooded animal including human in need thereof a therapeutically effective amount of compounds of formulas I-XXIII.

The magnitude of the therapeutic or prophylactic dose of the compounds of the present invention in the treatment or prevention of T2DM, pathological consequences of T2DM, inhibition of amyloidosis and prevention of pancreatic β-cell death depends upon severity and nature of the condition being treated and the route of administration. The dose and the frequency of the dosing will also vary according to age, body weight and response of the individual patient. In general the total daily dose range for a compound of the present invention is from approximately 0.1 to approximately 500 mg in single or repeated doses.

Any suitable routes of administration may be employed to provide an effective dosage of the compounds of the present invention. Possible routes are not limited by oral, intravenous, topical and parenteral administrations, with oral administration representing a preferred route.

Compounds of the present invention may be administered in association with one or more inert carriers, excipients and diluents forming a pharmaceutical composition. Certain preferred oral compositions contain between approximately 0.1% and approximately 75% of compounds of formulas I-XXIII.

Solid compositions for oral administration may include binders, such as syrups, acacia, sorbitol, polyvinylpyrrolidone, carboxymethylcellulose, ethyl cellulose, microcrystalline cellulose or gelatin and mixtures thereof; excipients, such as starch, lactose or dextrins; disintegrating agents, such as alginic acid, sodium alginate, primogel and the like; lubricants, such as magnesium stearate, heavy molecular weight acids such as stearic acid, high molecular weight polymers such as polyethylene glycol; sweetening agents, such as sucrose or saccharine; flavoring agents, such as peppermint, methyl salicylate or orange flavoring; and coloring agents.

The liquid pharmaceutical compositions of the invention, whether they are solutions, suspensions or other like form, may include sterile diluents such as water for injection, saline solution, preferably physiological saline, Ringer's solution, or isotonic sodium chloride, fixed oils such as synthetic mono or diglycerides which may serve as the solvent or suspending medium, polyethylene glycols, glycerin, propylene glycol or other solvents.

Suitable pharmaceutically acceptable salts of compounds II-III include salts of basic elements such as sodium, potassium, calcium and magnesium, with the preferred basic addition being a sodium salt.

The following Examples are offered by way of illustration and not by way of limitation.

### EXAMPLES

### Example 1

### Synthesis of 1H-Indole-2-Carboxamide (VIa)

Indole-2-carboxylic acid (10.26 g, 63.7 mmol) is dissolved in dichloromethane (125 ml) and oxalyl chloride (39.8 mL of a 2.0 M solution in methylene chloride) is slowly added dropwise to the reaction at room temperature. Upon full addition, dimethylformamide (0.32 mL) is added and the reaction is stirred for two hours. After two hours, the reaction solution is transparent yellow in color. Ammonia gas is then bubbled into the reaction for 25 minutes and the reaction is stirred at room temperature for an additional 30 minutes. The reaction then is partitioned between water and ethylacetate. The organic phase is washed with saturated ammonium chloride then is dried and concentrated to provide crude amide (9.53 g).

### Example 2

### Synthesis of 8-Hydroxy-7-Quinoline Carboxamide (Va)

8-Hydroxy-7-quinolinecarboxylic acid methyl ester is prepared according to Eckstein Z et al. (Pol. J. Chem. 1979, 53(11):2373-7). The ester is reacted with excess ammonia in a steel bomb for 12-18 hr. The excess ammonia is allowed to evaporate and the residue is crystallized from a suitable solvent to yield the title compound.

### Example 3

### Synthesis of Quinoxaline-2-Carboxamide (IVa)

To a suspension of the quinoxaline-2-carboxylate (542 mg, 3.13 mmol) in methanol (20 ml) is added 28% aqueous ammonia (1.5 ml), and the mixture is refluxed for 3 hours. Water is added to the residue obtained by distilling off solvent under reduced pressure and the precipitate is collected by filtration. After air-drying, these are dissolved into ethyl acetate, which is dried over anhydrous sodium sulfate. Solvent is removed by distillation, and the residue is decanted with isopropyl ether and air-dried to give the title compound (369 mg; yield 76%).

### Example 4

### Synthesis of 4H-Quinolizin-4-One-3-Carboxamide (VIIa)

a) To a solution of 2-methylpyridine (7 ml) in tetrahydrofuran (140 ml) is added dropwise a solution of n-butyl lithium (49 ml of 1.59 mol solution in hexane) with ice-cooling. The resulting dark red solution is allowed to warm to ambient temperature and is stirred for an hour. After cooling to −78° C., a solution of diethyl ethoxymethylenemalonate (15.68 ml) in tetrahydrofuran (50 ml) is added over a period of 30 minutes. The reaction mixture is allowed to warm to −20° C. and stirred for 30 minutes at −20° C. Acetic acid (4.48 ml) is added. The solvent is distilled off, the residue is dissolved in ethyl acetate and washed with 10% aqueous solution of sodium bicarbonate, water and saturated aqueous sodium chloride. After drying over magnesium sulfate, the ethyl acetate extract is filtered and evaporated to give an oil (27 g). The residue is chromatographed on silica gel (Merck 70-230 mesh, 270 g) eluting with chloroform to give ethyl 3-ethoxy-2-ethoxycarbonyl-4-(2-pyridyl)butyrate (19 g) as an oil.

b) A mixture of ethyl 3-ethoxy-2-ethoxycarbonyl-4-(2-pyridyl)butyrate (18.9 g), diphenyl (48.85 g) and diphenyl ether (135.8 g) is heated to 250° C. for 40 minutes. The reaction mixture is cooled to ambient temperature and chromatographed on silica gel (Merck 70-230 mesh, 620 g) eluting with hexane and then a mixture of ethanol and chloroform (1:49) to give a crude oil, which is crystallized from a mixture of ether and hexane (1:1) to give 3-ethoxycarbonyl-4H-quinolizin-4-one (11.48 g) as a yellow crystal.

c) To a solution of 3-ethoxycarbonyl-4H-quinolizin-4-one (2.17 g) in methanol (65.2 ml) is added dropwise 6 N aqueous sodium hydroxide (6.5 ml) at room temperature. After stirring for 20 minutes, water (10 ml) is added. After stirring for 20 minutes, water (30 ml) is also added. After stirring for an hour, the reaction mixture is acidified to pH 3 with 4N aqueous hydrochloric acid. The precipitate is filtered and washed with water to give 4H-quinolizin-4-one-3-carboxylic acid (1.75 g) as pale yellow crystal (m. p. 233° C.).

d) To a suspension of 4H-quinolizin-4-one-3-carboxylic acid (1.69 g) in methanol (80 mL) is added 25% of ammonia and the mixture is refluxed for 4 hours. Water is added to the residue obtained by distilling off solvent under reduced pressure and the precipitate is collected by filtration. After air-drying, these are dissolved into ethyl acetate, which is dried over anhydrous sodium sulfate. Solvent is distilled off, the residue is decanted with isopropyl ether and air-dried to obtain 1.3 g of the 4H-quinolizin-4-one-3-carboxamide.

### Example 5

### Synthesis of 3-(N-Methylamido)-N-Methyl Phenyl Propanamide (Ia)

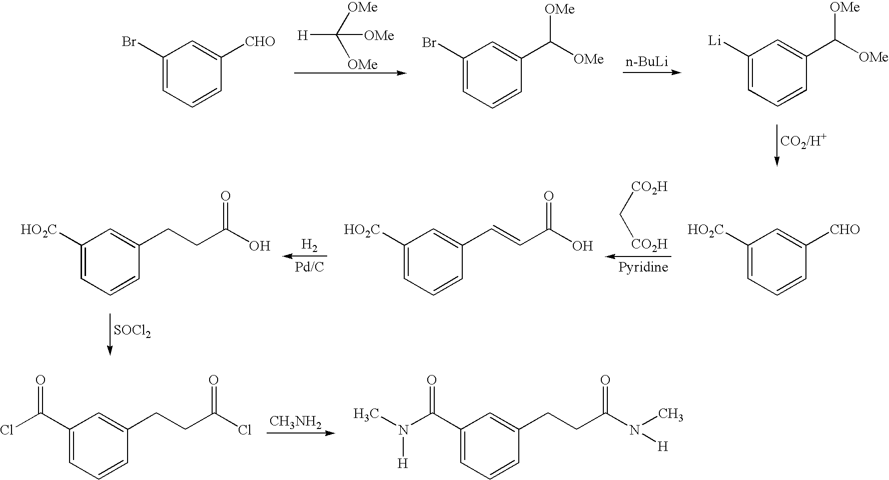

### Step-1:

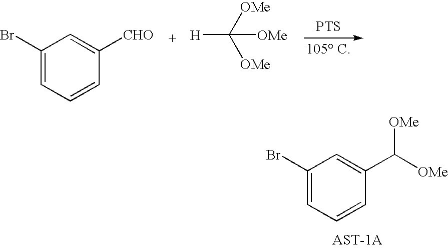

10 gm 3-Bromobenzaldehyde is taken in a 100 ml round bottomed flask. To this is added 20 ml trimethyl orthoformate and 100 mg p-toluene sulphonic acid. Reaction mixture is then refluxed for 2 hours. TLC shows formation of the product. Heating is stopped, and the reaction mixture is extracted with hexane (3×500 ml), washed with sodium bicarbonate solution (5%) to remove the traces of p-toluenesulphonic acid. Reaction mixture is then dried over sodium sulphate and concentrated to give 12 gm dimethyl acetal of 3-bromobenzaldehyde (AST-1A).

### Step-2:

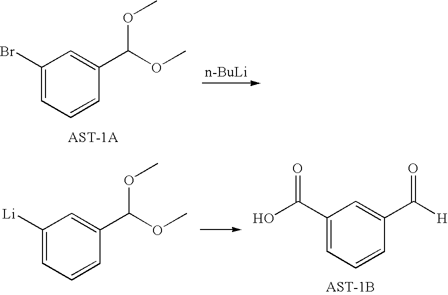

5 gm AST-1A is taken in 10 ml THF in a 3-necked round-bottomed flask and kept under nitrogen atmosphere in a tub containing dry ice to maintain the temperature at about −60° C. It is then stirred for 40 minutes. 3 gm dry ice is taken in a beaker and the reaction mixture is poured on dry ice with stirring. After complete addition, cold water and dilute hydrochloric acid are added to reaction mixture to a pH of 4. Then it is extracted with ethyl acetate (2×500 ml), dried over sodium sulfate and concentrated to give solid product (1.4 gm). Washing with hexane to remove impurities shown by PMR gives a final yield of 1.1 gm AST-1B.

### Step-3

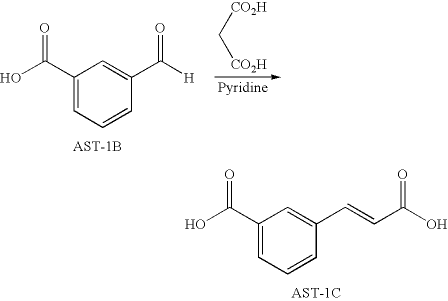

Malonic acid (1 gm) and pyridine (3 ml) is taken in a 100 ml RBF. It is kept in cold under stirring. To this is added 1 gm AST-1B (3-formyl benzoic acid). The reaction mixture is stirred overnight at room temperature. Then it is heated for 1 hour, cooled and quenched in water, and extracted with ethyl acetate (2×500 ml). The ethyl acetate layer is washed with dilute hydrochloric acid to pH 4.The ethyl acetate layer is then dried over sodium sulphate and concentrated to yield the product (1 gm).

### Step-4

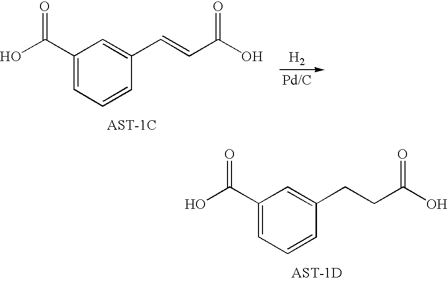

10% Palladium on charcoal (500 mg) is added to 50 ml methanol in a round-bottomed flask and connected to a hydrogen cylinder through a trap. To this round bottomed flask is added 4 gm AST-1C and hydrogen gas is bubbled overnight. Stirring and hydrogen bubbling is stopped. The reaction mixture is filtered through Celite. The filtrate is collected, and methanol is removed by distillation under vacuum to give solid product (4 gm) AST-1D. Reduction of the unsaturated compound is confirmed by NMR.

### Step-5

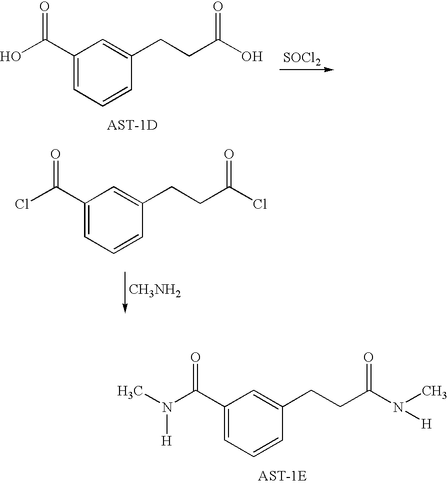

1 gm AST-1D is placed in a 3-necked round-bottomed flask set with condenser and magnetic stirrer. To this is added 5 ml thionyl chloride, and the material is refluxed for 1 hour. Formation of acid chloride is confirmed by derivatizing it to ester and checking TLC. Excess thionyl chloride is then removed by distillation. 10 ml methylamine (liquefied) is placed in another round-bottomed flask maintained at −8° C. It is then stirred for half an hour and allowed to attain room temperature. The solid formed is filtered off, and the product obtained in the filtrate is concentrated and subjected to column chromatography using DCM and methanol to isolate 0.4 gm AST-1E=Ia (final product).

### Example 6

### Synthesis of Sodium Salt of Iminostilbene-2-Sulfonate (IIIa)

Iminostilbene (5.0 g, 24.5 mmol) (Acros Organics) in acetic anhydride (7.1 mL, 75.1 mmol) and acetic acid (25 mL) is refluxed for 20 h. After cooling to room temperature, the reaction mixture is diluted with water (200 mL). The resulting solids are filtered, washed with water and dried to give N-acetyl iminostilbene as a white solid (5.2 g, 79%).

A flask containing 5 g (22.4 mmol) of N-acetyl iminostilbene in 200 mL of carbon tetrachloride is stirred and cooled to −7° to 0° C. while 2.66 g (25 mmol) of chlorosulfonic acid is added dropwise. As the chlorosulfonic acid is added, the product precipitates. The content must be stirred because the mixture is very thick. The supernatant solvent is decanted and to the residue is added 400 mL of water. Most of the solid dissolves and solution is filtered. A layer of carbon tetrachloride separates and is rejected. The solution is hydrolyzed with potassium hydroxide and the precipitate is filtered and dried to give 7.5 g of N-acetyl iminostilbene-2-sulfonate.

7.5 g (20 mmol) of N-acetyl iminostilbene-2-sulfonate in 100 ml of ethanol and 23.1 g (350 mmol) of potassium hydroxide is heated to a gentle reflux for 24 h at 90° C. under nitrogen. The batch is subsequently stirred into 1 liter of ice water and the mixture is extracted with dichloromethane. The organic phase is washed with water and dried over sodium sulphate. The solvent is removed by distillation in vacuo. The reaction product, sodium salt of iminostilbene-2-sulfonate (IIIa), remaining as a residue, is purified by column chromatography on silica gel using methanol/triethylamine, 95:5, as eluent.

### Example 7

### Synthesis of 2-Oxo-2,3-Dihydrobenzooxazole-3-Methylsulfonamide (XIVb)

A solution of 2-nitrophenol (3.38 g, 20 mmol) in absolute THF (80 mL) containing 10% Pd—C-catalyst (100 mg) is hydrogenated at ambient temperature and pressure until the calculated amount of H₂ has been taken up. To the resultant colorless solution are added with stirring and external cooling and under exclusion of O₂ (N₂ atmosphere), triethylamine (in one portion; 4.04 g, 40 mmol) and then rapidly a solution of bis(trichloromethyl carbonate) (2.0 g, 6.7 mmol) in THF (20 mL). After 30 min Et₃N.HCl and the catalyst are removed by suction, and THF is completely removed from the filtrate under reduced pressure. The pale brown crystalline residue is dissolved in boiling benzene (200 mL) and this solution is filtered while hot through a filter aid of 4 mm silica gel (0.063-0.200 mm). The filter pad is washed with hot benzene (150 mL). Cooling of the filtrate then affords 2-oxo-2,3-dihydroxybenzoxazole as colorless needles that are isolated by suction; yield: 2.5 g (75%); m. p. 154-155.

The 2-oxo-2,3-dihydroxybenzoxazole is dissolved into methylene chloride (185 mL) and added to triethylamine (10 mL, 69 mmol) in a 500 mL 3-neck flask fitted with a thermometer under a nitrogen atmosphere. The mixture is then cooled to 0° C. and methanesulfonyl chloride (5.0 mL, 41 mmol) is added by syringe. The mixture is permitted to come to room temperature, and stirred overnight, under a nitrogen system. The reaction is quenched with excess water, and the organic layer is dried with anhydrous sodium sulfate, filtered, and concentrated under vacuum, yielding 5.03 g viscous oil. Purification is conducted using a Hewlett-Packard HPLC 2000, with two silica cartridges, and eluting with a 1:1 hexane:ethyl acetate solvent system, yielding the final title compound (3.5 g, 38%) as a white solid.

### Example 8

### Synthesis of 4-Carboxamido-4-Oxazoline-2-One-3-Methylsulfonamide (IXa)

3-[(p-nitrobenzenesulfonyl)oxy]-2-oxopropanoate is synthesized by the methodology of Hoffman R V et al. (J. Org. Chem. 1997, 62:2458-65). A mixture of 3-[(p-nitrobenzenesulfonyl)oxy]-2-oxopropanoate (0.30 g, 0.99 mmol), methyl carbamate (0.37 g, 5.0 mmol) and p-toluenesulfonic acid monohydrate (0.019 g, 0.1 mmol) in 20 ml of toluene is refluxed overnight. The reaction is monitored by TLC (EtOAc/CH₂Cl₂, 1:9). The reaction mixture is cooled to room temperature, 80 ml of EtOAc is added, and the mixture is washed with water (2×60 mL) and brine (60 mL), dried (MgSO₄) and concentrated in vacuo to provide a yellow solid. The crude product is chromatographed on a silica gel column eluting with hexanes/EtOAc (gradient of 4:1 then 2:1) and recrystallized from EtoAc/hexanes to provide 4-carbomethoxy-4-oxaxazolin-2-one as a white crystalline solid (0.15 g, 51%), m.p. 150-152° C.

To a stirring solution of 4-carbomethoxy-4-oxaxazolin-2-one in dichloromethane (0.1 M) at 0° C. under nitrogen is added triethylamine (3.5 eq) and methanesulfonyl chloride (1.1 eq). The reaction is stirred at room temperature overnight. The reaction mixture is washed with saturated sodium bicarbonate (1 time), brine (1 time), dried over sodium sulfate, filtered, concentrated in vacuo and purified by flash chromatography using the ISCO system (0-15% gradient methanol/dichloromethane) to afford 4-carbomethoxy-4-oxaxazoline-2-one-3-methylsulfonamide.

To a solution of 4-carbomethoxy-4-oxaxazolin-2-one-3-methylsulfonamide (1g) in methylene chloride (10 mL) is added ammonia methanol solution (60 mL) that is prepared by bubbling the ammonia gas (14 g) into methanol (120 mL), and the mixture is stirred for hours at ambient temperature. After evaporating the solvent the residue is recrystallized from the methanol to give 4-carboxamido-4-oxaxazoline-2-one-3-methylsulfonamide (0.78 g).

### Example 9

### Synthesis of N-Methylsulfonylformamide (XVIIa)

Maleimide is obtained from the Sigma Aldrich Chemical Co. N-methylsulfonylmaleimide is obtained by sulfonation, as in Example 7, as a white powder, 0.45 g.

### Example 10

### Synthesis of N-Methylsulfonyl-2,4,5-Imidazolidinetrione (XXIIa)

2,4,5-imidazolidinetrione is obtained from Sigma Aldrich Chemical Co. N-methylsulfonyl-2,4,5-imidazolidinetrione is obtained by the sulfonation, as in Example 7, as white powder, 0.7 g.

### Example 11

### Amyloid Binding Assay

All test articles and control drugs are added (final concentrations 1-100 μM) to the wells of a 96-well plate (10 mM phenol red free Tris-HCl, pH 7.4) and incubated for 30 minutes at ca 37° C. in humidified 5% CO₂ atmosphere, followed by the addition of cytotoxic target (IAPP) (final concentration 25 μM) to the appropriate wells. Thioflavin T (final concentration 5 μM) is then added to the appropriate wells immediately following the addition of CTT. The plate is mixed gently on a gyratory shaker, incubated at ca 37° C. in a humidified 5% CO₂ atmosphere and read directly on a Tecan Safire reader in the fluorescence mode at excitation 450 nm and emission at 482 nm at 0, 1, 3, and 6 hours. Experiments are run in duplicate on each of two plates.

### Example 12

### Cell Culture and Cytotoxicity Assays

RINm5F cells are cultured in RPMI 1640 medium containing 10% fetal bovine serum, 290 μg/ml 1-glutamine, 100 units/ml penicillin and 100 μg/ml streptomycin (Aitken et al. 2003). Cells are plated in 24-well plates at a density of 15×10⁴ cells per well, incubated for 48 h, rinsed with PBS and placed in fresh medium (200 μl/well) in the presence or absence of compounds I-VIII (final concentrations 0.1 1, 10 and 100 μM). Following 30 minutes incubation, 12 μl of a freshly prepared aqueous solution of human IAPP (500 μM) is added to the cell culture medium to give final human IAPP concentration of 28 μM. Following 22h, cell viability is determined by double staining with calcein-AM and EthD-1. Green fluorescence of live cells and red fluorescence marking nuclei of dead cells are simultaneously visualized using a Zeiss axiovert S100 microscope equipped with a Zeiss filter set#09. Photographs are taken at 400× magnification using a Zeiss AxioCam digital camera. The EC₅₀ for the inhibition of cytotoxicity of IAPP by compounds I-VIII ranges from 0.6 μM to 100 μM.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A pharmaceutical composition for the treatment of type 2 diabetes mellitus (T2DM), pathological consequences of T2DM, or inhibition of IAPP-induced amyloidosis, or the prevention of death of pancreatic β-cells, comprising a pharmaceutical carrier, diluent or excipient and an effective amount of a compound of formula I: 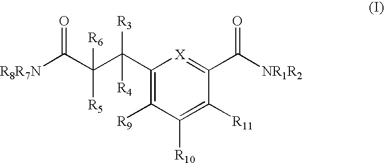
wherein X is C—H fragment or nitrogen;
R₁, R₂, R₇, R₈ are independently selected from hydrogen and C₁-C₃ alkyl;
R₃, R₄, R₅, R₆ are independently selected from hydrogen, methyl, ethyl and propyl;
R₉, R₁₀, R₁₁ are independently selected from bromine, chlorine, fluorine, carboxy, hydrogen, hydroxyl, hydroxymethyl, methanesulfonamido, nitro, sulfamyl, trifluoromethyl, C₂-C₇ alkanoyloxy, C₁-C₆ alkyl, C₁-C₆ alkoxy, C₂-C₇ alkoxycarbonyl, C₁-C₆ thioalkyl, aryl, CON(R₃₈R₃₉) and N(R₃₈R₃₉) wherein R₃₈ and R₃₉ are independently selected from hydrogen, acetyl, methanesulfonyl and C₁-C₆ alkyl; and
with the proviso that aromatic carbon atoms may be optionally replaced by aromatic nitrogen atoms.
